(19) United States
(12) Patent Application Publication
(10) Pub. No.: US 2010/0030169 A1
(43) Pub. Date: Feb. 4, 2010

(54) Catheter system

(76) Inventors: Tonya Dauterman, Fostoria, OH (US); Kurt Dauterman, Fostoria, OH (US)

Correspondence Address: MARSHALL & MELHORN, LLC, FOUR SEAGATE - EIGHTH FLOOR, TOLEDO, OH, 43604, US

(21) Appl. No.: 12/462,171

(22) Filed: Jul. 30, 2009

Related U.S. Application Data
(60) Provisional application No. 61/137,344, filed on Jul. 30, 2008.

(51) Int. Cl. A61F 5/44

(52) U.S. Cl. 604/328

(57) ABSTRACT

A catheter retaining system for a container is disclosed. The system may be utilized with a wide variety of containers for receiving catheterized fluids. Each embodiment of the system includes a structure for selectively securing at least one catheter to the container. The structure assists in maintaining the catheter in the proper location with respect to the container to minimize or eliminate spills, prevent contamination of sterile environments or the specimen, facilitate the catheterization process for patients and prevent patient embarrassment.

### RELATED APPLICATION

This application is claiming the benefit, under 35 U.S.C. §119(e), of the provisional application filed Jul. 30, 2008 titled Catheter System under 35 U.S.C. §111(b), which was granted Ser. No. 61/137,344. This provisional application is hereby incorporated by reference.

### FIELD OF THE INVENTION

The present invention relates to a catheter system designed to provide a quickly made but secure connection between the system and any liquid receiving container.

### BACKGROUND OF THE INVENTION

A catheter is a tube that can be inserted into a body cavity, duct or vessel. Catheters allow drainage, injection of fluids or access by surgical instruments. In most uses a catheter is a thin, flexible tube (“soft” catheter), although in some uses it is a larger, solid tube (“hard” catheter). The catheter may be left inside the body either temporarily or permanently.

Frequently, catheters are used to drain urine from a bladder. The process of inserting one end of the catheter in one's body, while maintaining the other end in a container for receiving the urine, can be difficult for one person to do on their own. Even when the catheter is already inserted in a patient's body, the inserted end may fall out of the container as the patient moves, the container moves, the tube moves or when catheterized fluid begins to flow through the tube. Typically, this is because there is no mechanism for maintaining the container end of the catheter in the container. When the container end leaves the container, urine, or other fluids, spill in beds, on cloths, on the patient and on health care providers. Such spills are highly undesirable as they can lead to bacterial contamination of sterile environments or urine specimens and patient embarrassment.

In view of the above disadvantages of permitting the end of the catheter to remain unsecured in the container, it would be advantageous to have the end secured. It would be further advantageous for a catheter security device to be easy to use, inexpensive, capable of being used by a single person and readily adaptable to current bodily fluid containers.

### SUMMARY OF THE INVENTION

In one embodiment, a catheter retaining system comprises a container for receiving catheterized fluids. A conical end piece receiving portion is located on an inside surface of a mouth portion of the container. The end piece receiving portion has an inner surface shaped to selectively secure an end piece of a catheter therein. The conical end piece receiving portion may be also located on alternative containers, as described herein.

In another embodiment, a flexible hook-like structure is located on the inside surface of the mouth portion. The structure selectively extends at least partially about an end piece of a catheter to selectively secure the catheter within the mouth portion. The structure may be also located on alternative containers, as described herein.

In yet another embodiment, the system comprises a lid that at least partially selectively closes a fluid collection portion of a container. An aperture is located through the lid where the aperture selectively secures an end piece of a catheter.

### BRIEF DESCRIPTION OF THE DRAWINGS

The above, as well as other advantages of the present invention, will become readily apparent to those skilled in the art from the following detailed description when considered in the light of the accompanying drawings in which:

FIG. 1A is a schematic depiction of a front view of the present invention located in a urinal bottle;

FIG. 1B is a schematic view of the present invention;

FIG. 2 is a schematic, partial, cut-away side view of the invention of FIG. 1;

FIG. 3 is a schematic, partial, cut-away side view of the invention of FIG. 2;

FIG. 4 is a schematic depiction of a front view of another embodiment of the present invention;

FIG. 5 is a plan, schematic view of another embodiment of the invention;

FIG. 6 is a plan, schematic view of a variation on the invention of FIG. 5;

FIGS. 7A and 7B are schematic front and side views of another embodiment of the invention;

FIGS. 8A and 8B are schematic front and side views of another embodiment of the invention;

FIGS. 9A and 9B are schematic front and side views of another embodiment of the invention; and

FIGS. 10A and 10B are schematic front and side views of another embodiment of the invention.

### DETAILED DESCRIPTION OF THE PREFERRED EMBODIMENTS

It is to be understood that the invention may assume various alternative orientations and step sequences, except where expressly specified to the contrary. It is also to be understood that the specific devices and processes illustrated in the attached drawings, and described in the following specification are simply exemplary embodiments of the inventive concepts defined herein. Hence, specific dimensions, directions or other physical characteristics relating to the embodiments disclosed are not to be considered as limiting, unless expressly stated otherwise.

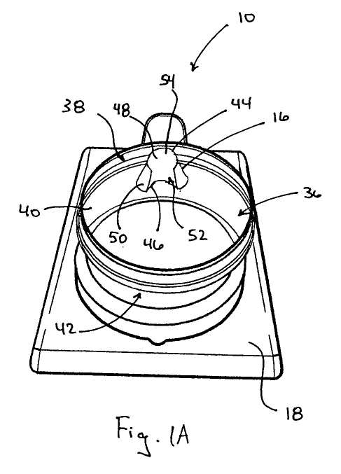

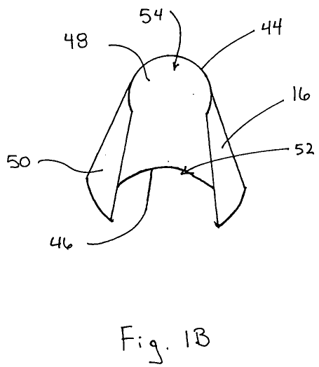

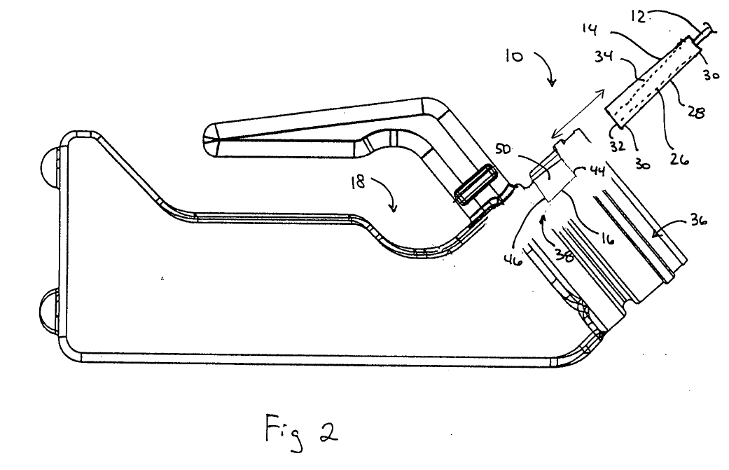

Turning now to FIGS. 1, 2 and 3, one embodiment of the present invention is depicted. This embodiment comprises a catheter system 10 having a drain tube 12, an end piece 14 and an end piece receiving portion 16 located within a container. The container may be, but is not limited to, a bottle-type urinal 18. A bottle-type urinal 18 is used herein merely for illustrative purposes.

The container may comprise any receptacle capable of collecting and holding a fluid. Containers within the scope of the invention may comprise, but are not limited to, bottles, plastic urinals, cups, or trays. As can be appreciated herein, the invention may work with containers without further modification or the container may be adapted for convenient use with the invention.

The fluid may comprise any known fluid but is likely to be bodily fluids, such as urine or blood, whether from a human or an animal.

The drain tube 12 may be such as surgical tubing which typically is both relatively small in diameter and flexible. A channel 20 extends continuously from a first end 22 of the tube 12 to a second end 24 of the tube 12.

At, or proximate, the first end 22 of the tube 12, at least one aperture (not shown) is provided. There may be more than one aperture at the first end 22 and the aperture or apertures may be oriented and located about the first end 22 as known by those skilled in the art.

The end piece 14 is attached to the second end 24 of the tube 12. Preferably, the end piece 14 is securely attached to the second end 24 such that it will not easily become detached from the tube 12. Alternatively, the end piece 14 may be unitary and one-piece with the tube 12. The tube 12 may terminate anywhere within the end piece 14.

It is preferred that the end piece 14 has a means for permitting fluid to flow through it. Such means may comprise, but are not limited to, a hollow channel 26 substantially coaxial with the tube 12. The hollow channel 26 may have a design complimentary to an exterior surface 28 of the end piece 14, or it may have a different design.

The exterior surface 28 of the end piece 14 may be such as a frusto conical design, such as a tapered cone. In this embodiment, a first end 30 having a smaller diameter is connected to a second end 32 having a larger diameter. A continuous body portion 34 connects the first end 30 with the second end 32.

The end piece 14 may be constructed of a plastic or rubber-like material that is preferably somewhat elastic, although rigid designs are also permissible.

While one shape of the end piece 14 has been described, the present invention is not limited to such shapes and others may be within the scope of the present invention. By way of example only, the continuous body portion 34 may be substantially uninterrupted, as shown, or it may have steps or grooves. Further, the end piece may also comprises a body portion with a substantially constant outer diameter.

The end piece receiving portion 16 is preferably located within a mouth portion 36 of the bottle urinal 18. While the figures depict one bottle urinal design, those skilled in the art will readily appreciate that there are numerous other types of such designs, all of which are within the scope of the present invention. Further, the present invention is not limited to locating the end piece receiving portion 16 in a bottle-type urinal; instead, it may be used in conjunction with any container, such as those mentioned above.

Preferably, the end piece receiving portion 16 is located on an upper portion 38 of the mouth portion 36 as this may assist in maintaining the end piece receiving portion 16 out of fluid collected in the bottle 18. While one location of the end piece receiving portion 16 is shown in the figures, it can be appreciated that other locations are permissible. For example, the end piece receiving portion 16 might be located on a side wall 40 of the mouth portion 36 or on a bottom portion 42 of the mouth portion 36. It is also permissible for the mouth portion 36 to have one or more end piece receiving portions 16 adjacent one another or spaced apart from one another about the mouth portion 36.

In one embodiment of the end piece receiving portion 16 depicted in FIGS. 1, 2 and 3, the end piece receiving portion 16 is of a frusto-conical, or tapered cone, design that is complimentary to the shape of the end piece 14. More specifically, the end piece receiving portion 16 may have a first end 44, a second end 46, an inner surface 48 and an outer surface 50. The inner surface 48 defines a substantially hollow interior portion 52. A slot 54 may be located from the first end 44 to the second end 46.

The first end 44 has a diameter that is smaller than a diameter of the second end 46. The inner and outer surfaces 48, 50 may be parallel to one another, but need not be. In the preferred embodiment, the end piece receiving portion 16 defines a gradually and continuously expanding inner and outer surfaces 48, 50 from the first end 44 to the second end 46. It is also within the scope of the present invention for the end piece receiving portion to have a substantially constant outside diameter.

The end piece receiving portion 16 may be separately formed from and then selectively attached to the mouth portion 36 of the bottle 18. By way of example only, the end piece receiving portion 16 may be selectively secured to the mouth portion 36 by tape, adhesive, hook and loop type fasteners, complimentary male and female connectors, clips or clamps and/or mechanical fasteners or it may be made part of the collection device itself.

In the depicted embodiment, the end piece 14 has a complimentary shape to the end piece receiving portion 16. More specifically, the frusto-conical shape of the exterior surface 28 of the end piece 14 is slightly smaller than, but in the same shape as, the frusto-conical shape of the inner surface 48 of the end piece receiving portion 16. Thus, it can be appreciated that the end piece receiving portion 16 may selectively receive the end piece 14 therein.

Preferably, the end piece receiving portion 16 is shaped so as to selectively lock the end piece 14 therein, thus portion 16 and end piece 14 selectively interlock with one another. The design of the end piece 14 described above, thus permits it to be located securely within the end piece receiving portion 16 but also permits it to be released from the end piece receiving portion 16.

In the depicted embodiment, the frusto-conical design of the end piece receiving portion 16 is oriented such that the first, smaller end 44 is adjacent the mouth portion 36 opening and the second, larger end 46 is adjacent the more significant volume of the bottle. Therefore, the smaller end 30 of the end piece 14 can be located into the smaller end 44 of the end piece receiving portion 16 and the larger end 32 of the end piece 14 can be located within the larger end 46 of the end piece receiving portion 16.

It can be appreciated that the tube 12 can be located through the slot 54 of the end piece receiving portion 16 such that it can then extend through the open end of the larger end 46 of the end piece receiving portion 16.

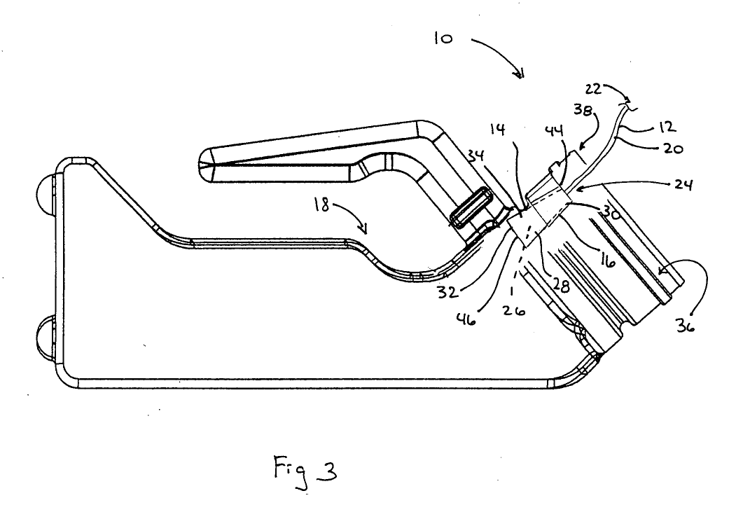

FIG. 4 depicts yet another embodiment of a catheter system 55 within the scope of the present invention. In one embodiment, an end piece receiving portion 56 is integrally formed and unitary with a mouth portion 58 of a bottle 60. This end piece receiving portion 56 forms a slot 62 within the mouth portion 58 for receiving a drain tube end piece, which is not shown but it is as depicted and described above. The slot 62 connects with a substantially continuous hollow interior 64 of the portion 56 to accept the end piece.

The portion 56 is depicted as being located at a top portion 66 of the bottle 60, however, the portion 56 may be located anywhere in the mouth portion 58. Further, more than one portion 56 may be located in the mouth portion 58 for receiving more than one end piece.

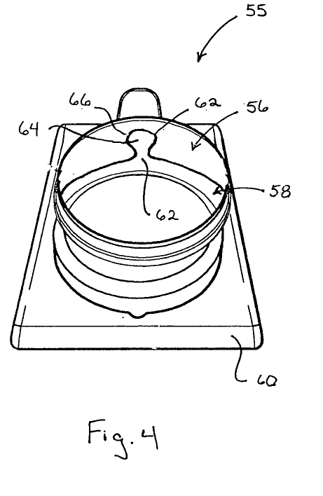

FIG. 5 depicts yet another embodiment of the present invention wherein the end piece 14 described above is inserted into an end-piece receiving portion 68 located on another type of fluid container. In this case, the fluid container is a tray 70. The tray 70 of FIG. 5 is substantially rectangular, but it may be of any design, including oval, circular, rectangular and/or about any other geometric design without limitation.

The end-piece receiving portion 68 is preferably of a frusto-conical, or tapered cone, design that is complimentary to the shape of the end piece 14. More specifically, the end piece receiving portion 68 may have a first end 74, a second end 76, an inner surface 78 and an outer surface 80. The inner surface 78 defines a substantially hollow interior portion 82 in the shape of a channel. A slot 84 may be located from the first end 74 to the second end 76. The slot 84 may define the portion 68 in to two upstanding walls 85′ and 85″.

The first end 74 has a diameter that is smaller than a diameter of the second end 76. The inner and outer surfaces 78, 80 may be parallel to one another, but need not be. In the preferred embodiment, the end piece receiving portion 68 defines a gradually and continuously expanding inner and outer surfaces 78, 80 from the first end 74 to the second end 76.

The end-piece receiving portion 68 may be separate from the tray 70. The portion 68 may be selectively attached to the tray 70 by tape, adhesive, hook and loop type fasteners, complimentary male and female connectors, clips or clamps and/or mechanical fasteners. Alternatively, portion 68 may be integrally formed and unitary with a portion of the tray 70. By way of example, the portion 68 may be integrally formed from and unitary with a side 72 of the tray 70, as shown in FIG. 5.

The portion 68 may be located anywhere on or in the tray 70. In addition, while only a single portion 68 is depicted in FIG. 5, it can be appreciated that more than one portion 68 may be located on or in the tray 70. Regardless of its location on or in the tray 70, the end piece, such as item 14 described above and depicted in FIGS. 1-3, may be engaged with the portion 68 to selectively secure the end piece 14 to the portion 68.

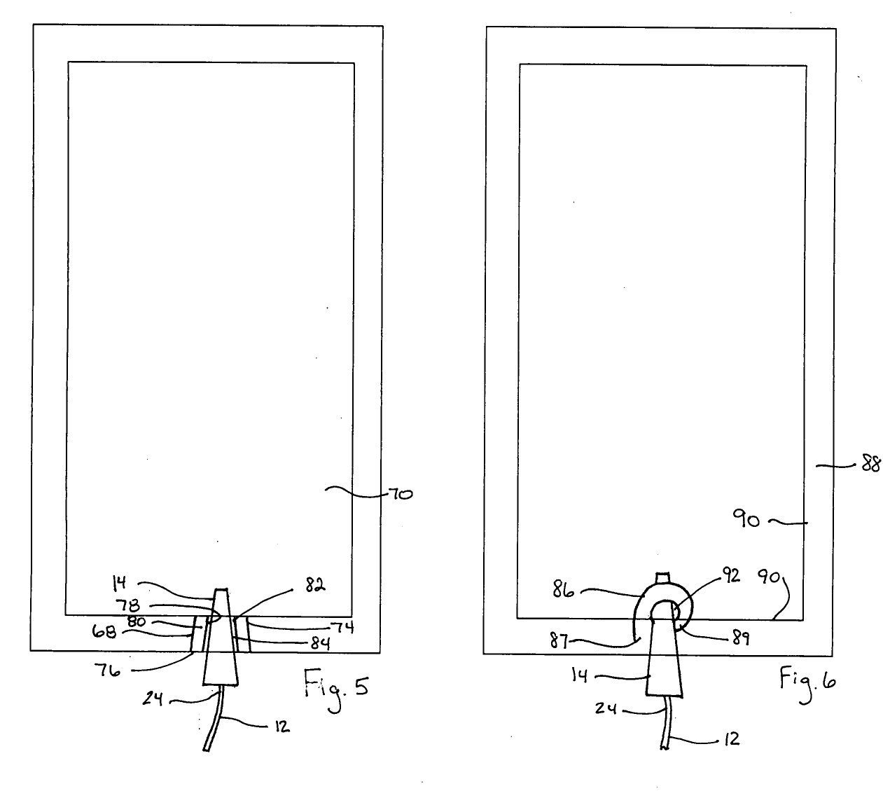

FIG. 6 depicts another embodiment of the present invention. In this embodiment, a curvilinear end piece receiving portion 86 is utilized to secure the end piece 14 of a catheter. As in FIG. 5, the portion 86 is depicted located on a tray 88, however, the present invention also includes locating the portion 86 on trays of various sizes and shapes (as stated above) as well as on bottle-type urinals, such as that described and depicted above, any other fluid collection devices (also mentioned above).

The portion 86 may be separate from the tray 88. The portion 88 may be selectively attached to the tray 88 by tape, adhesive, hook and loop type fasteners, complimentary male and female connectors, clips or clamps and/or mechanical fasteners. Alternatively, portion 86 may be integrally formed and unitary with any portion of the tray 88. By way of example, the portion 86 may be integrally formed from and unitary with a side 90 of the tray 88, as shown in FIG. 6.

The portion 86 may be located anywhere on or in the tray 88. In addition, while only a single portion 86 is depicted in FIG. 6, it can be appreciated that more than one portion 86 may be located on or in the tray 88. Regardless of its location on or in the tray 88, the end piece, such as item 14 described above and depicted in FIGS. 1-3, may be engaged with the portion 86.

The portion 86 is preferably substantially curvilinear such that it forms a curlicue-type design. Preferably, the portion 86 curls in on itself so that it forms a selectively openable, central aperture 92 for receiving and retaining the end piece 14. The portion 86 may be formed of any material, but preferably it is a flexible material than can be urged open, the end piece 14 can be inserted in the aperture 92 and then the portion 86 substantially returns to its original curvilinear design about the end piece 14 to secure the end piece 14 therein.

The curlicue portion 86 may have a connection portion 87 adjacent the container it is attached to. The portion 86 may or may not gradually taper to an end portion 89.

The portion 86 may extend entirely about the end piece 14 one or more times, or the portion 86 may extend only partially about the end piece 14. Regardless, the portion 86 may take on other forms as well with a more geometric design. For example, the interior and exterior surfaces may be substantially planar so as to form a J-shape or hook-type design.

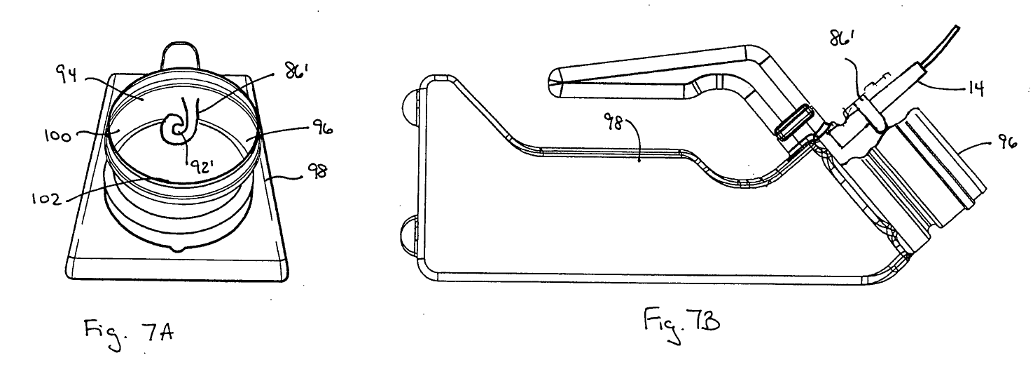

FIGS. 7A and 7B depict the portion 86′ within a bottle type urinal 98, which has been described above. The portion 86′ is substantially similar to portion 86 described above.

Preferably, portion 86′ is located on an upper portion 94 of the mouth portion 96 as this may assist in maintaining the portion 86′ out of fluid collected in the bottle 98. While one location of the portion 86′ is shown in the figures, it can be appreciated that other locations are permissible. For example, the portion 86′ might be located on a side wall 100 of the mouth portion 96 or on a bottom portion 102 of the mouth portion 86′. It is also permissible for the mouth portion 96 to have one or more portions 96 adjacent one another or spaced apart from one another about the mouth portion 96.

The portion 86′ may be separately formed from and then selectively attached to the mouth portion 96 of the bottle 98. By way of example only, the portion 86′ may be selectively secured to the mouth portion 96 by tape, adhesive, hook and loop type fasteners, complimentary male and female connectors, clips or clamps and/or mechanical fasteners or it may be made part of the collection device.

FIG. 7B depicts an end piece 14, such as described above, engaged with the portion 86′. The end piece 14 is inserted into a central aperture 92′, and the portion 86′ wraps at least partially about the end piece 14. The portion 86′ selectively holds the end piece 14 so that fluid can be drained into the bottle 98.

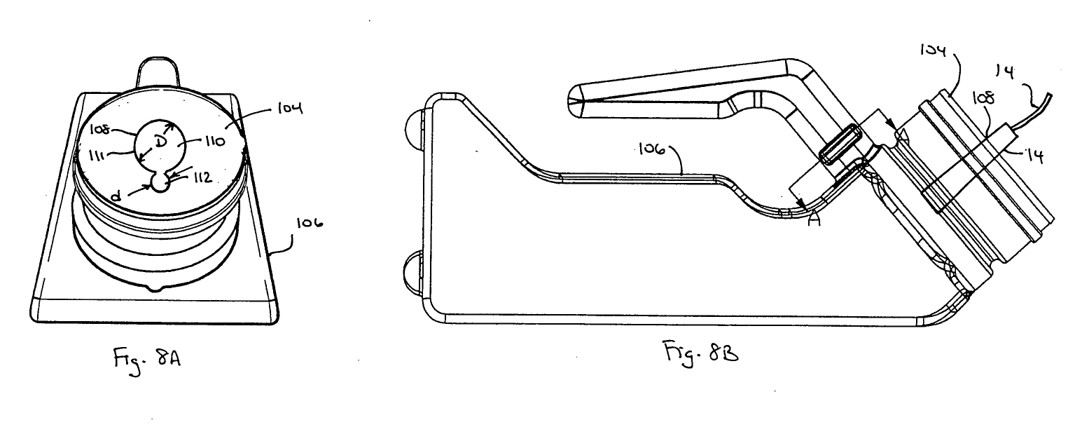

FIGS. 8A and 8B depict yet another embodiment of the present invention, wherein a selectively removable lid 104 is provided on a bottle 106, such as a bottle type urinal described above. The lid 104 may be securely, but removably, engaged with the bottle 106. The lid 104 may be formed of a polymeric material that preferably has both resilience and flexibility.

An aperture 108 may be located within the lid 104. The aperture 108 is depicted as being located substantially centered in the lid 104, but it may be located anywhere in the lid 104. The aperture 108 may be comprised of two portions—a main portion 110 and a minor portion 112. In this embodiment, the main portion 110 and the minor portion 112 are connected to one another and share a common perimeter 111.

The main portion 110 may have a larger diameter D than a diameter d of the minor portion 112. The larger diameter D of the main portion 110 facilities locating the end piece 14 of the catheter into the aperture 108. Once the end piece 14 is located within the lid 104, the drain tube 14 may be dropped down into the minor portion 112. The end piece 14 can then be partially withdrawn through the minor portion 112 so that the exterior surface 28 of the end piece 14 can be selectively secured by inner circumference 114 of the minor portion 112.

To withdraw the end piece 14 from the minor portion 112, the end piece 14 may be urged slightly into the bottle 106 so that the inner diameter 114 is no longer in gripping contact with tapered exterior surface 28 of the end piece 14. The end piece 14 may then be lifted out of the aperture 108.

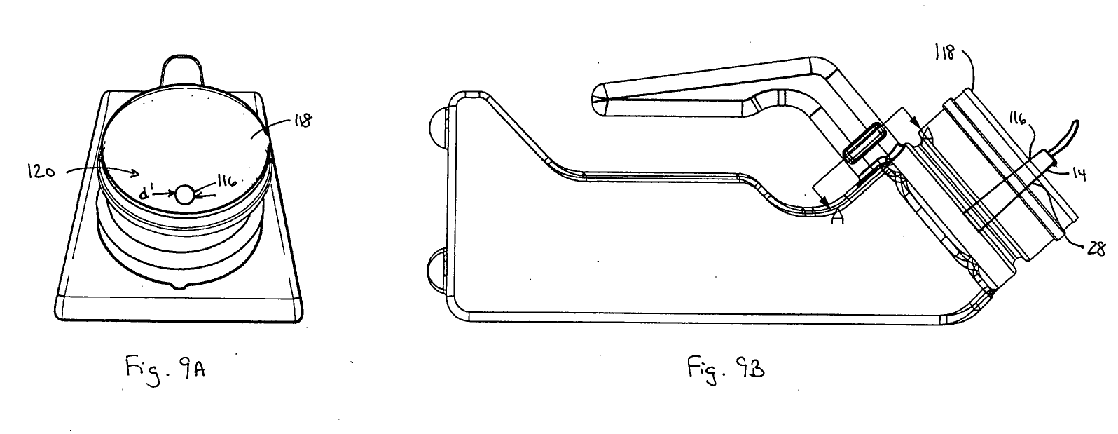

FIGS. 9A and 9B depict yet another embodiment of the present invention. In this embodiment, at least one aperture 116 is provided in a lid 118. The lid 118 may be substantially as disclosed above, except for the cited differences. While the aperture 116 may be located anywhere in the lid 118, in the depicted embodiment, the aperture 116 is located in a lower portion 120 of the lid 118.

The aperture 116 has a diameter d′ that approximates the outer diameter of the exterior surface 28 of the end piece 14. As shown in FIG. 9B, the end piece 14 may be selectively inserted into the aperture 116 to be secured within the lid 118. The flexible nature of the lid 118 permits the end piece 14 to be removed therefrom by pulling the end piece 14 through the lid 118. The aperture 116 expands as the larger end of the end piece 14 is withdrawn through it. The aperture 116 then returns substantially to its original size.

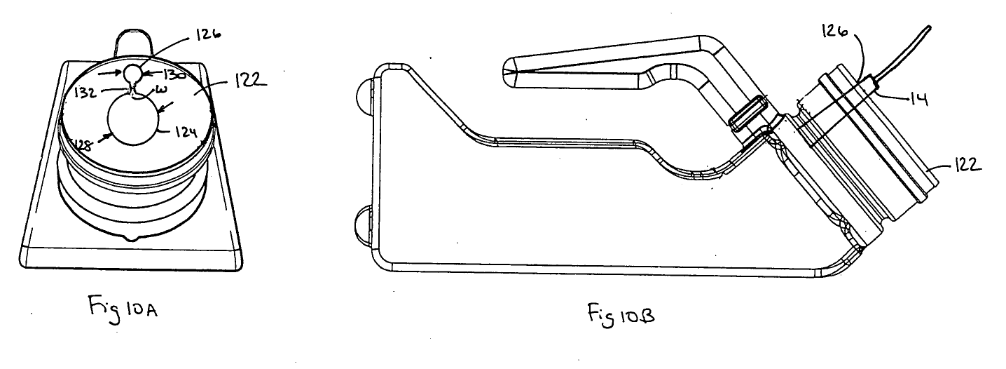

FIGS. 10A and 10B depict yet another embodiment of the present invention wherein a selectively removable lid 122 is provided on a container, such as a bottle type urinal described above. The lid 122 may have two apertures 124, 126. A first aperture 124 may have a larger diameter 128 than a diameter 130 of the second aperture 126, however, this is not a requirement of the present invention. Further, FIG. 10A depicts the second aperture 126 above the first aperture 124. Again, this is not a requirement of the present invention and the second aperture 126 may be located in any relationship about the first aperture 124.

Preferably, a slot 132 connects the first aperture 124 and the second aperture 126. The slot 132 may have a width W that is less than both of the diameters 128,130 of the apertures 124,126.

The end piece 14 described above may interlock with the lid 122 by locating it initially through the larger diameter aperture 124. The tube 12 may be slid up the slot 132 until it is located in the aperture 126. Next, the end piece 14 may be withdrawn from the bottle through the aperture 126 until the diameter 130 of the second aperture 126 securely retains the end piece 14 therein.

In accordance with the provisions of the patent statutes, the present invention has been described in what is considered to represent its preferred embodiments. However, it should be noted that the invention can be practiced otherwise than as specifically illustrated and described without departing from its spirit or scope.

What is claimed is:

1. A catheter retaining system for a container, comprising:
a container for receiving catheterized fluids, said container having a mouth portion; and
a conical end piece receiving portion located on an inside surface of said mouth portion; said end piece receiving portion having an inner surface shaped to selectively secure an end piece of a catheter therein.

2. The system of claim 1, wherein said end piece receiving portion is one-piece and unitary with said inner surface.

3. The system of claim 1, wherein said end piece receiving portion is selectively attached to said inner surface.

4. The system of claim 1, wherein said end piece receiving portion has a slot extending from a first end to a second end, said slot in communication with a channel that extends entirely through said end piece receiving portion.

5. The system of claim 4, wherein said end piece receiving portion has a conical outer surface and a conically shaped inner surface defining said channel.

6. The system of claim 4, wherein said slot is substantially opposite a connection portion of said end piece receiving portion and said mouth portion.

7. The system of claim 1, wherein said end piece receiving portion is located substantially on an upper portion of said mouth portion.

8. The system of claim 1, wherein said container is a bottle-type urinal.

9. The system of claim 1, wherein said end piece of said catheter has a substantially complementary shape to said end piece receiving portion.

10. A catheter retaining system for a container, comprising:
a container for receiving catheterized fluids therein, said container having a hollow mouth portion; and
a flexible curlicue structure located on an inside surface of said mouth portion, said structure selectively extending at least partially about an end piece of a catheter to selectively secure said catheter within said mouth portion.

11. The system of claim 10, wherein said curlique structure is one-piece and unitary with said container.

12. The system of claim 10, wherein said curlique structure is selectively attached to said container.

13. The system of claim 10, wherein said curlique structure wraps at least partially about a circumference of said end piece.

14. The system of claim 10, wherein said curlique structure is tapered from a connection portion at said mouth portion to an end portion.

15. The system of claim 1, wherein said curlique has a curlicue with a central aperture for receiving said end piece.

16. A catheter retaining system for a container, comprising:
a container for receiving catheterized fluids therein, said container having a fluid collection portion and a perimeter portion at least partially surrounding said fluid collection portion; and
a conical end piece receiving portion located on said perimeter portion, said end piece receiving portion having an inner surface shaped to selectively interlock with an end piece of a catheter therein.

17. The system of claim 16, wherein said end piece receiving portion is connected to said perimeter portion above said fluid collection portion of said container.

18. The system of claim 16, wherein said inner surface of said end piece receiving portion defines at least two upstanding walls separated by a slot, said walls together form a channel for said end piece.

19. A catheter retaining system for a container, comprising:
a container for receiving catheterized fluids therein, said container having a fluid collection portion and a perimeter portion at least partially surrounding said fluid collection portion;
a flexible curlicue structure located on said perimeter portion, said structure extending at least partially about an end piece of a catheter to selectively secure said end piece to said perimeter portion.

20. The system of claim 19, wherein said curlicue has a central aperture for receiving and wrapping at least partially about said end piece.

21. A catheter retaining system for a container, comprising:
a container for receiving catheterized fluids therein, said container having a lid that at least partially selectively closes a fluid collection portion of said container; and
at least a first aperture located through said lid, said first aperture having a circumference that extends at least partially about an end piece of a catheter to selectively secure said end piece of said catheter and said lid.

22. The system of claim 21, wherein said lid comprises a second aperture, said second aperture having a larger diameter than a diameter of said first aperture and wherein said apertures at least partially share a common circumference.

23. The system of claim 21, wherein said lid has a central aperture, and wherein said at least one radial slot extends from said central aperture through said lid to said first aperture.
